(19) United States
(12) Patent Application Publication
(10) Pub. No.: US 2026/0125420 A1
(43) Pub. Date: May 7, 2026

(54) METHOD AND SYSTEM FOR INCREASING PURITY OF PROTEIN FROM A GRAIN PROTEIN RECOVERY SYSTEM

(71) Applicant: Fluid Quip Technologies, LLC, Cedar Rapids, IA (US)

(72) Inventors: David Koziol, Fort Calhoun, NE (US); Christopher Kempf, Cedar Rapids, IA (US)

(21) Appl. No.: 19/373,245

(22) Filed: Oct. 29, 2025

Related U.S. Application Data
(60) Provisional application No. 63/714,925, filed on Nov. 1, 2024.

(51) Int. Cl. C07K 1/34; C07K 14/415; C12N 1/06; C12R 1/645

(52) U.S. Cl. CPC C07K 1/34; C07K 14/415; C12N 1/063; C12R 2001/645

(57) ABSTRACT

A method and system for increasing the purity of protein from a system that produces protein, such as a high protein meal, from dry milling of grains including, for example, corn and wheat. The purity of the high protein meal, for example, can be increased by back end enzyme treatment of yeast, which has been separated along with protein from whole stillage. The enzyme(s) can breakdown the yeast into its yeast components that can be removed from the protein to provide for an increased purity of a resulting high protein product.

### TECHNICAL FIELD

The present invention relates to dry grind methods and systems of alcohol production and increasing the purity of protein from systems that separate protein from a whole stillage byproduct in a plant that derives a biochemical, such as alcohol (e.g., ethanol), from grain.

### BACKGROUND

Dry milling ethanol plants generally convert corn and/or other grains into three products, i.e., ethanol, distillers grain oil, and distiller's grains with solubles. A typical grain dry milling process consists of four major steps: grain handling and milling, liquefaction and saccharification, fermentation and distillation, and co-product recovery. Grain handling and milling is the step in which the grain is brought into the plant and ground to promote better conversion of starch to glucose. Liquefaction is the step of converting solids, such as starch, to a flowable liquid producing oligosaccharides and saccharification is where the oligosaccharides are converted into single glucose molecules. Fermentation is the process of yeast or bacteria, or as clostridia, for example, converting glucose into a biofuel or a biochemical/biomolecule, such as ethanol. Distillation is the process of removing the biofuel or biochemical/biomolecule, such as ethanol, from the fermentation product. Co-product recovery is the step in which the grain by-products are de-watered and made ready for market. There are many known chemical and biological conversion processes known in the art that utilize yeast, bacteria, or the like to convert glucose/sugar to other biofuels and biochemical/biomolecule components like ethanol, for example.

The recovery of alcohol, e.g., butanol, ethanol (a natural co-product), etc., and other similar compounds, generally begins with the beer (spent fermentation broth) being sent to a distillation system. With distillation, ethanol is typically separated from the rest of the beer through a set of stepwise vaporizations and condensations. The beer less the alcohol extracted through distillation is known as whole stillage, which contains a slurry of the spent grains including grain protein, fiber, oil, minerals, sugars, and fermentation agent. This byproduct is too diluted to be of much value at this point and is further processed to provide the distiller's grains with solubles.

In typical processing, when the whole stillage leaves the distillation column, it is generally subjected at the back end of the process to a decanter centrifuge to separate insoluble solids or “wet cake”, which includes mostly fiber, from the liquid or “thin stillage”, which includes, e.g., protein, fine fiber, oil, and amino acids. After separation, the thin stillage moves to evaporators to boil away moisture, leaving a thick syrup that contains the soluble (dissolved) solids. The concentrated syrup is typically mixed with the wet cake, and the mixture may be sold to beef and dairy feedlots as distillers wet grain with solubles (DWGS). Alternatively, the wet cake and concentrated syrup mixture may be dried in a drying process and sold as distillers dried grain with solubles (DDGS). The resulting DDGS generally has a crude protein content of about 29% and is a useful feed for cattle and other ruminants due to its protein and fiber content.

In addition, in some processes currently used to produce high protein grain meal, including those that separate protein and yeast cells from fiber and germ fragments of a whole stillage stream, the separated protein (and yeast) meal stream can include a total protein concentration that can be hindered by the inclusion of the yeast in the protein meal stream. That is, the presence of yeast negatively impacts the total concentration of the protein and can prevent any resulting protein grain meal product from achieving higher more desirable protein concentrations.

While DDGS and DWGS provide a critical secondary revenue stream that offsets a portion of the overall ethanol production cost, it would be beneficial to provide a method and system where a high protein grain product can be obtained, such as with a more desirable or increased purity level, from the whole stillage to be sold at a higher cost per ton than DDGS or DWGS or than other low/lower purity protein grain products.

### SUMMARY OF THE INVENTION

The present invention is directed to a method and system for increasing the purity of protein from a system that produces protein, such as a high protein meal, from dry milling of grains including, for example, corn and wheat. Here, the purity of a high protein meal, for example, can be increased by enzyme treatment of yeast, which has been separated along with protein from whole stillage. The enzyme(s) can breakdown yeast bodies into its yeast components that can be removed from the protein to provide for an increased purity of a resulting high protein product.

In one embodiment, a method for increasing the purity of protein from a whole stillage byproduct from a biochemical process includes separating whole stillage into an insoluble solids portion and a filtrate portion via a pressure screen followed by subjecting the filtrate to a first decanter centrifuge to separate out solids, including yeast, from the centrifuged filtrate portion. The solids, including the yeast, can be sent to an enzyme treatment tank whereat one or more enzymes can break down the yeast cell wall thereby breaking up the yeast and releasing its contents, which can be later removed from the solids portion. Thereafter, the enzymatically treated solids portion can be subjected to a second decanter centrifuge to remove the enzyme(s) therefrom as centrate, which can be recycled back in the system and reused at the enzyme treatment tank. The resulting centrate from the first decanter centrifuge then can be recombined with the solids, including protein and yeast components or fragments, from the second decanter centrifuge. This process allows for yeast fragments to be later removed from the protein in the combined centrate and solids stream via subsequent protein separation methods/apparatuses. This setup can provide a more desirable/increased protein purity in a final or resulting protein grain meal product.

In another embodiment, a method for increasing the purity of a protein product obtained from a whole stillage byproduct produced in a biochemical production process includes separating a whole stillage byproduct into a fiber portion and a filtrate, which includes protein particles and yeast cells, and then subjecting the filtrate to one or more enzymes to break-up the yeast cells into yeast fragments. Next, the enzymatically treated filtrate, including the protein particles and yeast fragments, is separated, via density, into a protein portion, including protein particles, and a water soluble solids portion, including yeast fragments and free oil. Then, the protein portion can be dried to define a high protein grain meal that includes from 40 wt % protein to 80 wt % protein on a dry basis.

In another embodiment, a method for increasing the purity of a protein product obtained from a whole stillage byproduct produced in a biochemical production process includes separating a whole stillage byproduct, via filtration, into a fiber portion and a filtrate, which includes protein particles and yeast cells, and then separating the filtrate, via density, into a first solids portion, including the protein particles and yeast cells, and a first centrate. Next, the first solids portion is subjected to one or more enzymes to break-up the yeast cells into yeast fragments. Then, the enzymatically treated first solids portion are separated, via density, into a second solids portion, including the protein particles and yeast fragments, and a second centrate, including the one or more enzymes, which are recycled back into the method and reused as at least a portion of the one or more enzymes. Thereafter, the second solids portion, including the protein particles and yeast fragments, is separated, via density, into a protein portion, including protein particles, and a water soluble solids portion, including yeast fragments and free oil. Then, the protein portion is dried to define a high protein grain meal that includes from 40 wt % protein to 80 wt % protein on a dry basis.

In yet another embodiment, a system for increasing the purity of a protein product obtained from a whole stillage byproduct produced in a biochemical production process includes a first apparatus that is configured to receive a whole stillage byproduct produced in a biochemical production process, wherein the first apparatus separates the whole stillage byproduct into a fiber portion and a filtrate, which includes protein particles and yeast cells, and an enzyme treatment tank that is situated after the first apparatus and that is configured to receive one or more enzymes and the filtrate from the first apparatus, and whereat the yeast cells in the filtrate are broken-up into yeast fragments via the one or more enzymes. The system further includes a second apparatus that is situated after the enzyme treatment tank and that is configured to receive the enzymatically treated filtrate from the enzyme treatment tank, wherein the second apparatus separates the enzymatically treated filtrate, including the protein particles and yeast fragments, via density, into a protein portion, including protein particles, and a water soluble solids portion, including yeast fragments and free oil, and a dryer that is situated after the second apparatus and that is configured to receive and dry the protein portion to define a high protein grain meal that includes from 40 wt % protein to 80 wt % protein on a dry basis.

### BRIEF DESCRIPTION OF THE DRAWINGS

The accompanying drawings, which are incorporated in and constitute a part of this specification illustrate embodiments of the invention and, together with the general description of the invention given above and the detailed description given below, serve to explain the principles of the invention. Similar reference numerals are used to indicate similar features throughout the various figures of the drawings.

FIG. 1 is a flow diagram of a typical dry grind alcohol production process;

FIG. 2 is a flow diagram of a prior art method and system for producing a high protein meal from a whole stillage byproduct produced via a dry milling process of grain for making ethanol;

FIG. 3 is a flow diagram of a method and system for increasing the purity of protein via enzyme treatment of yeast from whole stillage in a dry grind biochemical process in accordance with an embodiment of the invention; and

FIG. 4 is a flow diagram of a method and system for increasing the purity of protein via enzyme treatment of yeast from whole stillage in a dry grind biochemical process in accordance with another embodiment of the invention.

### DETAILED DESCRIPTION OF SPECIFIC EMBODIMENTS

The present invention relates to dry grind methods and systems of alcohol and/or biochemical production for increasing the purity of protein from systems that separate protein (and yeast) from whole stillage in a plant that derives alcohol (e.g., ethanol) from corn, wheat, or other cereal grains. In particular, the back end of the alcohol and/or biochemical production process can produce a high protein meal stream from a whole stillage byproduct. The purity of the high protein meal can be increased by enzyme treatment of yeast, particularly yeast bodies, which has been separated along with grain protein from whole stillage. The enzyme(s) can breakdown the yeast into its yeast components that can be removed from the protein to provide for an increased purity of a resulting high protein product, as further discussed in detail hereinbelow.

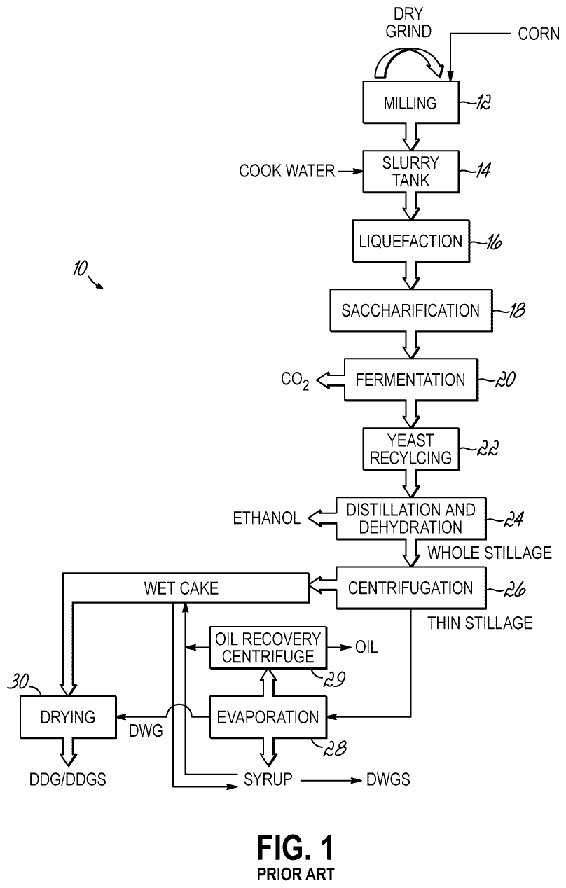

FIG. 1 shows a flow diagram of a typical dry grind alcohol (e.g., ethanol) production method 10. Although virtually any type and quality of grain, such as but not limited to sorghum, wheat, triticale, barley, rye, tapioca, cassava, potato, pea and other starch and/or oil containing grains and/or legumes can be used to produce ethanol and/or a biochemical/biomolecule, for example, the feedstock for this process is typically corn referred to as “No. 2 Yellow Dent Corn.” Also, as a general reference point, the dry grind method 10 can be divided into a front end and a back end. The part of the method 10 that occurs prior to distillation 24 is considered the “front end,” and the part of the method 10 that occurs after distillation 24 is considered the “back end.” To that end, the front end of the dry grind method 10 begins with a milling step 12 in which dried whole corn kernels can be passed through hammer mills for grinding/milling into meal or a fine powder. The screen openings in the hammer mills or similar devices typically are of a size 6/64 to 9/64 inch, or about 2.38 mm to 3.57 mm, but some plants can operate at less than or greater than these screen sizes. The resulting particle distribution yields a very widely spread, bell type curve, which includes particle sizes as small as 45 microns and as large as 2 mm to 3 mm. The majority of the particles tend to be in the range of 500 to 1200 microns, which is the “peak” of the bell curve. Other screen openings larger and smaller can be deployed in the hammer mills. Other milling devices such as roller mills or pin mills can also be utilized in the dry grain grinding step.

After the milling step 12, the ground meal is mixed with cook water to create a slurry at the slurry tank 14 and a commercial enzyme called alpha-amylase is typically added (not shown). Creating the slurry at the slurry tank 14 is followed by a liquefaction step 16 whereat the pH is adjusted to about 4.8 to 5.8 and the temperature maintained between about 50° C. to 105° C. so as to convert the insoluble starch in the slurry to soluble starch. The stream after the liquefaction step 16 has about 30% dry solids (DS) content, but can range from about 29-36%, with all the components contained in the corn kernels, including starch/sugars, protein, fiber, starch, germ, grit, oil, and salts, for example. Higher solids are achievable, but this requires extensive alpha amylase enzyme to rapidly breakdown the viscosity in the initial liquefaction step. There generally are several types of solids in the liquefaction stream: fiber, germ, and grit.

Liquefaction 16 may be followed by separate saccharification and fermentation steps, 18 and 20, respectively, although in most commercial dry grind ethanol processes, saccharification and fermentation can occur simultaneously. This single step is referred to in the industry as “Simultaneous Saccharification and Fermentation” (SSF). Both saccharification and SSF can take as long as about 50 to 60 hours. Gluco-Amylase enzyme is typically added to the fermentation step that facilitates the further breakdown of the starches and larger polysaccharides into single monomer sugar molecules that the yeast consumes to produce ethanol (or other similar alcohols) and carbon dioxide. Yeast can optionally be recycled in a yeast recycling step 22 either during the fermentation process or at the very end of the fermentation process. Yeast produced during the fermentation process will pass through to the distillation and dehydration step 24. In addition to the gluco-amylase being added, other enzymes can be added (such as but not limited to phytase, protease, cellulase, hemicellulose, xylanase, beta-glucanase, and the like) that can further enhance the protein and oil recovery downstream. Subsequent to the fermentation step 20 is the distillation (and dehydration) step 24, which utilizes a still to recover the alcohol (e.g., ethanol).

Finally, a centrifugation step 26 involves centrifuging the residuals, i.e., “whole stillage”, which includes the non-fermentable grain components (protein, oil, fiber, ash, and minerals, for example) and yeast yielded from the distillation and dehydration step 24 in order to separate the insoluble solids (“wet cake”) from the liquid (“thin stillage”). The liquid from the centrifuge contains about 5% to 12% DS. The “wet cake” includes fiber, of which there generally are three types: (1) pericarp, with average particle sizes typically about 1 mm to 3 mm; (2) tipcap, with average particle sizes about 500 micron; (3) and fine fiber, with average particle sizes of about 250 microns. There may also be proteins and yeast bodies with a particle size of about 45 microns to about 300 microns. The fiber and other fractions may contain bound protein that is chemically and or physically attached to the fiber and other fraction.

The thin stillage typically enters evaporators in an evaporation step 28 in order to boil or flash away moisture, leaving a thick syrup which contains the soluble (dissolved) solids (mainly protein and starches/sugars) from the fermentation (25 to 40% dry solids) along with residual oil and fine fiber. The concentrated slurry can be sent to a centrifuge to separate the oil from the syrup in an oil recovery step 29. The oil can be sold as a separate high value product. The oil yield is normally about 0.9 lb/bu of corn with elevated free fatty acids content compared to traditional wet mill corn oil. This oil yield recovers only about ½ of the oil in the corn, with part of the oil passing with the syrup stream and the remainder being lost with the fiber/wet cake stream. About one-half of the oil inside the corn kernel remains inside the germ after the distillation and dehydration step 24, which cannot be separated in the typical dry grind process using centrifuges as the oil is bound, not free. The free fatty acids content, which is created when the oil is heated and exposed to oxygen throughout the front and back-end process, reduces the value of the oil. The (de-oil) centrifuge only removes less than 50% because the protein and oil make an emulsion, which cannot be satisfactorily separated without the use of chemicals or added mechanical separation unit operations.

The syrup, which has more than 10% oil, can be mixed with the centrifuged wet cake, and the mixture may be sold to beef and dairy feedlots as Distillers Wet Grain with Solubles (DWGS). Alternatively, the wet cake and concentrated syrup mixture may be dried in a drying step 30 and sold as Distillers Dried Grain with Solubles (DDGS) to dairy and beef feedlots. This DDGS has all the corn and yeast protein and about 50% of the oil in the starting corn material. But the value of DDGS is low due to the high percentage of fiber, and in some cases the oil is a hindrance to animal digestion and lactating cow milk quality.

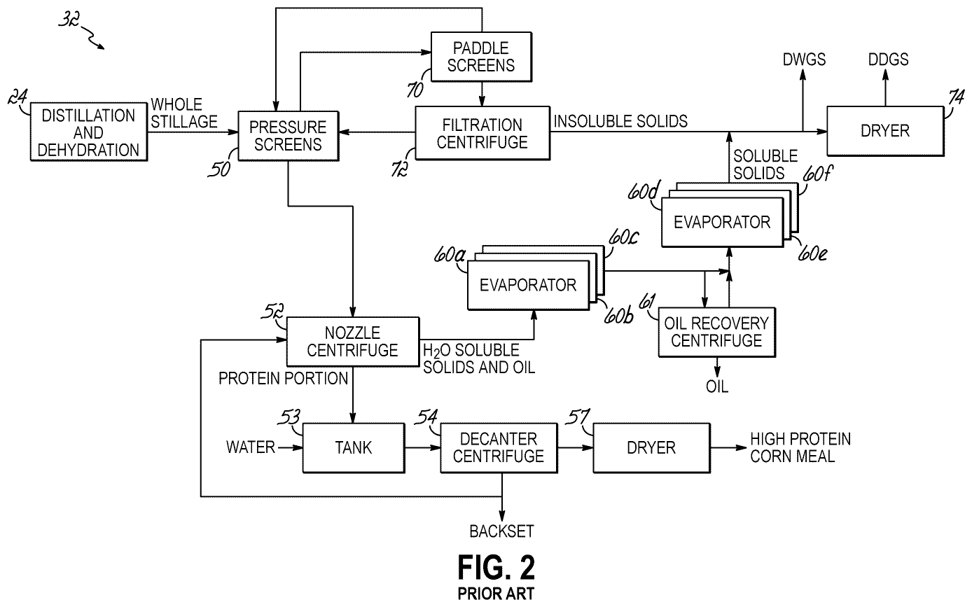

FIG. 2 shows a prior art method and system for producing a high protein corn meal, collectively numeral 32, from a whole stillage byproduct such as produced in a typical corn dry-milling method and system 10 like that just described in FIG. 1. The method begins with whole stillage from the distillation and dehydration step 24 of the dry grind ethanol method. The whole stillage is passed over one or more pressure screens 50 with openings sized to permit the passage of protein particles and yeast cells but reject/prevent the passage of most fiber particles.

The material that passes through the pressure screens in step 50 is higher in protein due, for example, to the exclusion of fiber by the pressure screens. This protein rich slurry or filtrate, which includes yeast cells, is sent to and further separated by one or more disc nozzle centrifuges in step 52, which can be arranged in series or parallel. In these centrifuges, the denser material including the protein particles and yeast can be concentrated in a high density underflow stream, which is referred to as a protein portion. The lighter material including most of the oil and some of the lighter (fine) fiber material passes to a lower density overflow stream. This overflow stream, which includes oil and water-soluble solids, is sent to a set of three evaporators 60a, 60b, and 60c, as are known in the art, whereat water is removed to begin to thicken the water soluble solids and oil stream to a high solids syrup.

Thereafter, the overflow stream can be piped and subjected to an optional oil recovery centrifuge 61 so that oil can be removed therefrom. In one example, the final recovered oil product can include between about 40 wt % to about 60 wt % of the total corn oil in the corn. The remainder of the overflow stream can be piped and subjected to another set of three evaporators 60d, 60e, and 60f whereat water is further evaporated to yield the high solids syrup. While FIG. 2 shows the overflow stream being subjected to two sets of three evaporators, 60a-c and 60d-f, it should be understood that the number of evaporators and sets thereof can be varied, i.e., can be more or less, from that shown depending on the particular application and result desired.

The underflow stream from the disc nozzle centrifuge 52, which defines the protein portion, is collected in a tank 53. Water can be added to the tank to dilute the underflow stream and help in washing soluble non protein solids from the high protein portion/slurry increasing the purity of a final protein meal. The underflow stream can be separated into a liquid fraction and a protein wet cake fraction at one or more decanting centrifuges 54 (e.g., a decanter centrifuge). The protein wet cake contains most of the protein as well as yeast and can be dried in a dryer 57 to make a high protein meal product. The liquid product or centrate is returned to the front end of the alcohol (e.g., ethanol) process as backset and used to slurry corn flour such as at slurry tank 14. In addition, at least a portion of the centrate may be returned back to the nozzle centrifuge 52.

The dewatered (and optionally dried) protein product can define a high protein meal that includes, for example, at least 40 wt % protein on a dry basis and which may be sold as pig or chicken feed. Because the high protein meal includes yeast along with its grain (e.g., corn) protein, the yeast can be a hindrance to realizing higher protein purity in any final protein meal product. The amount of yeast in the protein of the high protein meal can vary and, in one example, can include 24% of the total high protein meal. The range of the amount of yeast in the high protein meal generally can be from about 10% to about 35% depending upon the type of feedstock and other factors, including type of separation machinery, and the like, as would be understood by those skilled in the art.

Following the fiber stream exiting the pressure screen at 50, the high fiber wet cake that is rejected/separated by the pressure screen 50 can be (re)slurried and passed through one or more paddle screens 70. The wet cake that exits the paddle screens 70 can again be (re)slurried and then run through one or more filtration centrifuges 72. The centrate from both the paddle screens 70 and the filtration centrifuges 72 carries as yet unrecovered protein particles. These centrate streams are returned to the pressure screen 50 so that protein and oil that may have been missed on the first pass over the screens can be recovered by passing through the pressure screen 50 and on to the nozzle centrifuge 52.

The wet fiber cake that is separated from the liquid stream at the filtration centrifuge 72 is mixed with syrup from the evaporators 60f, 60e, 60d and is either sold as Distillers Wet Grains with Solubles (DWGS), or is run through a dryer 74 and is sold as Distillers Dried Grains with Solubles (DDGS).

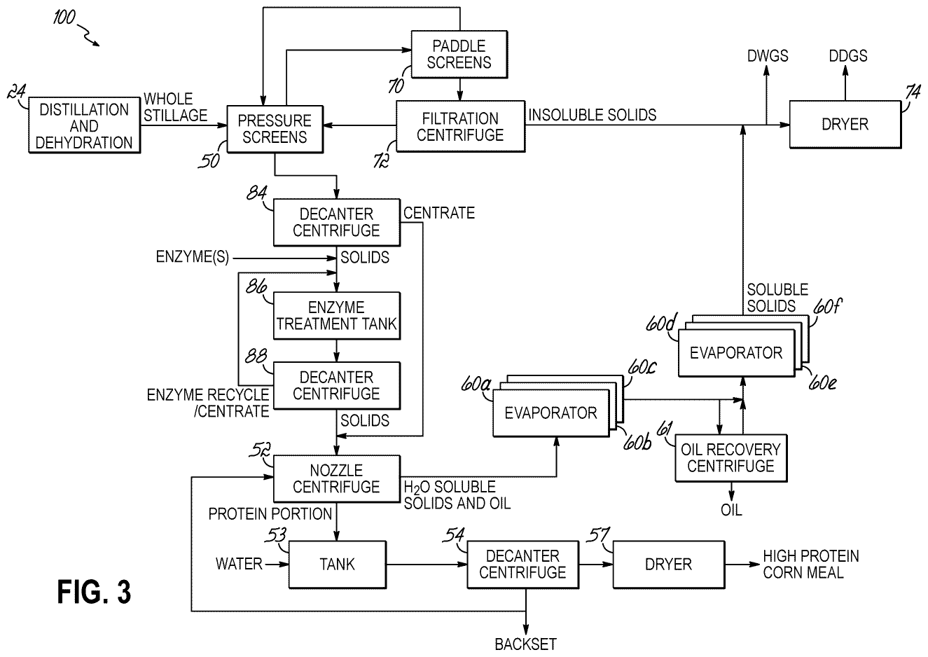

In accordance with the present invention, FIG. 3 shows one embodiment of a method and system for increasing the purity of protein in a dry grind process, collectively numeral 100, based, in part, on the dry grind process 32 just described in FIG. 2, with modifications/improvements made thereto. Generally, the purity of the high protein meal can be increased by enzyme treatment of yeast, which has been separated along with protein from the whole stillage. The enzyme(s) can breakdown the yeast into its yeast components that can be removed from the protein to provide for an increased purity of any resulting high protein product. The details of the modifications to FIG. 2 as set out in the embodiment of FIG. 3 are discussed hereinbelow. It is noted that certain reference numerals used in FIG. 2 are used here to represent like devices and/or steps in the method and system 100 of FIG. 3.

As shown in FIG. 3, in this embodiment, the protein rich slurry or filtrate from the pressure screens 50, which can include a significant fraction of the available protein of the grain feedstock from the whole stillage and a significant percentage of the dead yeast cells (yeast) from fermentation, can be sent and subjected to an optional first decanter centrifuge 84 to separate out solids, including yeast and protein, from the centrifuged filtrate portion to provide a separate centrate portion. It should be noted that the first decanter centrifuge 84 can include multiple centrifuges, either in series or parallel. One such suitable decanter centrifuge 84 is the SG806 available from Alfa Laval of Lund, Sweden. In one example, one or more of the first decanter centrifuge(s) 84 can be a solid bowl centrifuge or stacked-disc centrifuge or replaced by a filter press, a rotary drum vacuum filter, or other like device as is known in the art.

One or more enzymes can be mixed with the solids, which includes the yeast, from the first decanter centrifuge 84 and sent to an enzyme treatment tank 86 whereat the enzyme(s) can break down the yeast cell wall thereby breaking up the yeast and releasing its contents, which can be later removed from the solids portion. Although shown as mixing with the solids just prior to the enzyme treatment tank 86, it should be understood that the enzyme(s) may be mixed directly with the solids at the enzyme treatment tank 86 or even earlier in the method and system 100, such as between the pressure screens 50 and the first decanter centrifuge 84 or at the first decanter centrifuge 84, for example. The enzyme(s) can include a portion of optional recycled enzymes that have been recovered from the enzyme treatment tank 86 and sent back to mix again with the solids portion from the first decanter centrifuge 84, as further discussed below. In one example, the one or more enzymes can include amylase, alpha-amylase, glucoamylase, fungal, phytase, protease (e.g., serine protease), cellobiose, cellulase, hemicellulase, xylanase, glucanase, beta-glucanase, transglutaminase (e.g. microbial), zymolyase, and the like. In another example, the one or more enzymes can include a combination of cellulase and protease or xylanase and beta-glucanase.

The enzyme treatment tank 86 can be desirably sized to provide enough residence time for the enzyme(s) to break down the yeast. In one example, the enzyme treatment tank 86 can be sized so that the mean yeast cells residence time can be sufficient for the enzyme(s) to interact with and dissolve/break down the yeast cells (bulk of the yeast cells) into its components in the enzyme treatment tank 86. In one example, the enzyme treatment tank 86 can be a continuous stirred tank reactor (CSTR). Here, the solids portion and enzyme(s) can be both added and withdrawn from the CSTR at the same time on a continuous basis. The enzyme treatment tank 86 can be operated on level control, increasing or decreasing the amount of enzymatically treated solids moved or pumped out of the enzyme treatment tank 86 to match the amount of material entering the enzyme treatment tank 86, by means and methods known in the art.

The temperature and/or pH of the treated solids in the enzyme treatment tank 86 can be adjusted or controlled, by means and methods known in the art, to be a specified temperature and/or pH or within a certain range(s) to ensure desirable enzyme activity. The temperature and/or pH may need to be adjusted up or down. To control temperature, in one example, a heat exchanger can be provided either on the solids portion inlet flow to the enzyme treatment tank 86, i.e., between the first decanter centrifuge 84 and enzyme treatment tank 86, or on a recirculation line (coupled with a pump) associated with the enzyme treatment tank 86 such that the temperature of the incoming solids stream or enzymatically treated solids portion, respectively, can be controlled, such as by being lowered/cooled or raised/heated. If a heat exchanger is provided and not required, it may be set up to be bypassed, manually or automatically by means and methods known in the art. The optional heat exchanger may be a shell and tube or a plate and frame type heat exchanger, as are known in the art.

pH control may also be required, which can be in the form of a metering pump that can add a desired/required acid or base, for example, to the solids streams from the first decanter centrifuge 84 and/or the enzyme treatment tank 86 to ensure that a desirable pH or pH range is maintained to ensure desirable enzyme activity. In one example, the acid can include sulfuric acid, citric acid, hydrochloric acid, and the like, or the base can include sodium hydroxide, calcium carbonate, ammonia, and the like. The pH or pH range can vary depending on the enzyme(s) selected. In one example, the pH can be from 2 to 12. In another example, the pH can be from about 3 to 9. In another example, the pH can be from about 4 to 6.

After the enzyme treatment tank 86, as shown in FIG. 3, the enzymatically treated solids, with its protein and now broken down yeast or yeast components/fragments, can be subjected to an optional second decanter centrifuge 88 to remove the enzyme(s) as centrate from the centrifuged solids portion to provide a separate/remaining solids portion. The centrate, with its active enzyme(s), can be recycled back into the system, as shown, and reused again such as by being recombined with the solids from the first decanter centrifuge 84 to break down the yeast therein. Additional enzyme(s), as shown, may be needed to make up for enzyme lost with the solids portion from the second decanter centrifuge 88. It should be noted that the second decanter centrifuge 88 can include multiple centrifuges, either in series or parallel. One such suitable decanter centrifuge 88 is the SG806 available from Alfa Laval of Lund, Sweden. In one example, one or more of the decanter centrifuge(s) 88 can be a solid bowl centrifuge or stacked-disc centrifuge or replaced by a filter press, a rotary drum vacuum filter, or other like device as is known in the art.

The centrate from the first decanter centrifuge 84 then can be recombined with the solids stream, which includes protein and yeast components/fragments, from the second decanter centrifuge 88. The yeast fragments can be later removed from the protein in the combined centrate and solids stream via the subsequent protein separation methods/apparatuses, as described above in FIG. 2, which can provide a more desirable/increased protein purity in any final or resulting protein grain meal product. In particular and with continuing reference to FIG. 3, the combined centrate and solids stream can be sent to and separated by one or more disc nozzle centrifuges in step 52, which can be arranged in series or parallel. In these centrifuges, the denser material including the protein particles can be concentrated in a high density underflow stream, which is referred to as the protein portion. The lighter material including most of the oil, along with the yeast components, and some of the lighter (fine) fiber material passes to a lower density overflow stream. The percent protein in the underflow stream will now be enriched by the removal of the (lower protein) yeast cells. This will result in a higher purity protein product. The overflow stream, which includes oil, dissolved solids, and nutrients from the former yeast cells, is sent to the set of three evaporators 60a, 60b, and 60c, as are known in the art, whereat water is removed to begin to thicken the water soluble solids and oil stream to a high solids syrup. In one example, a portion of the overflow stream can be returned to the front end of the alcohol (e.g., ethanol) to slurry corn flour such as at slurry tank 14, which can provide additional nutrients to fermentation.

Thereafter, the overflow stream can be piped and subjected to the optional oil recovery centrifuge 61 so that oil can be removed therefrom. In one example, the final recovered oil product can include between about 40 wt % to about 60 wt % of the total corn oil in the corn. The remainder of the overflow stream can be piped and subjected to another set of three evaporators 60d, 60e, and 60f whereat water is further evaporated to yield the high solids syrup. While FIG. 3 shows the overflow stream being subjected to two sets of three evaporators, 60a-c and 60d-f, it should be understood that the number of evaporators and sets thereof can be varied, i.e., can be more or less, from that shown depending on the particular application and result desired.

The underflow stream from the disc nozzle centrifuge 52, which defines the protein portion, is collected in the tank 53. Water can be added to the tank to dilute the underflow stream and help in washing soluble non protein solids from the high protein portion/slurry further increasing the purity of a final protein meal. The underflow stream can be separated into a liquid fraction and a protein wet cake fraction at one or more decanting centrifuges 54 (e.g., a decanter centrifuge). The protein wet cake contains most of the protein and is without most of the yeast and can be dried in a dryer 57 to make a high protein meal product. The liquid product or centrate can be returned to the front end of the alcohol (e.g., ethanol) process as backset and used to slurry corn flour such as at slurry tank 14. In addition, at least a portion of the centrate may be returned back to the nozzle centrifuge 52.

The dewatered (and optionally dried) protein product, which excludes much of the yeast, can define a high protein meal that includes, for example, at least 40 wt % protein on a dry basis and which may be sold as pig or chicken feed. In another embodiment, the high protein meal includes at least 50 wt % protein on a dry basis. In another embodiment, the high protein meal includes at least 52 wt % protein on a dry basis. In yet another embodiment, the high protein meal includes at least 55 wt % protein on a dry basis. In still another embodiment, the high protein meal includes about 60, 70, or 80 wt %, protein on a dry basis. In another embodiment, the high protein meal can include from about 40 wt % to about 60, 70, or 80 wt % protein. In another embodiment, the high protein meal can include from about 50 wt % to about 60 wt % protein, about 52 wt % to about 60 wt % protein, or about 55 wt % to about 60 wt % protein on a dry basis.

The amount of yeast in the protein of the high protein meal here can vary and, in one example, can include 24% of the total high protein meal. The range of the amount of yeast in the high protein meal generally can be from about 5% to about 30% depending upon the type of feedstock and other factors, including type of separation machinery, and the like, as would be understood by those skilled in the art. Generally, the purity of the high protein meal can be increased by enzyme treatment of yeast. In one example, the purity of the high protein meal can be increased by 2% relative to a process without the enzyme treatment, for example, such as the process of FIG. 2. In another example, the purity of the high protein meal can be increased by 5%. In yet another example, the purity of the high protein meal can be increased by 10% or more. While the removal of yeast cells/yeast cell components from the protein stream may reduce the overall yield of the high protein meal, the removal can desirably increase the protein purity of the final product. That is, because the high protein meal now excludes yeast, the yeast is no longer a hindrance to realizing higher protein purity in any final protein meal product.

Following now the fiber stream exiting the pressure screen at 50, the high fiber wet cake that is rejected/separated by the pressure screen 50 can be (re)slurried and passed through one or more paddle screens 70. The wet cake that exits the paddle screens 70 can again be (re)slurried and then run through one or more filtration centrifuges 72. The centrate from both the paddle screens 70 and the filtration centrifuges 72 carries as yet unrecovered protein particles. These centrate streams are returned to the pressure screen 50 so that protein that may have been missed on the first pass over the screens can be recovered by passing through the pressure screen 50 and on to the nozzle centrifuge 52.

The wet fiber cake that is separated from the liquid stream at the filtration centrifuge 72 is mixed with syrup from the evaporators 60f, 60e, 60d, which includes additional yeast components, and is either sold as Distillers Wet Grains with Solubles (DWGS), or is run through a dryer 74 and is sold as Distillers Dried Grains with Solubles (DDGS).

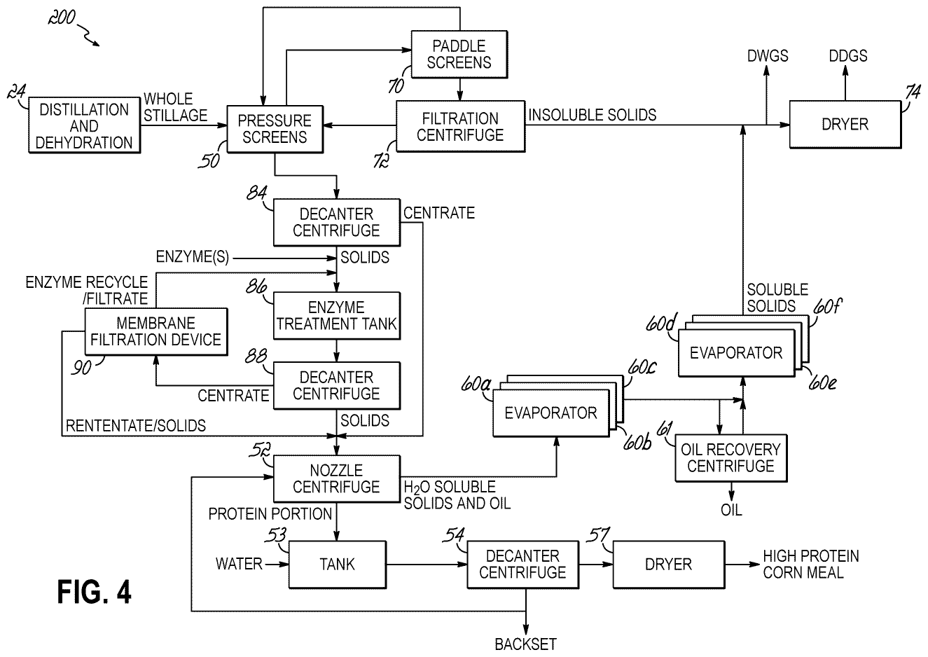

With reference to FIG. 4, another embodiment of a method and system for increasing the purity of protein in a dry grind process, collectively numeral 200, is shown that is similar to method and system 100 of FIG. 3, with the following modification(s). In this embodiment, as shown in FIG. 4, the centrate, with its active enzyme(s), from the second decanter centrifuge 88 is subjected to a membrane filtration device 90 to more thoroughly separate the various constituents in the enzyme recycle stream prior to recycling the enzyme(s) back for reuse. That is, the centrate from the second decanter centrifuge 88 is filtered, such as via the membrane filtration device 90, to provide a clean(er) enzyme filtrate or recycle stream and a separate retentate or solids portion, which includes solid particles and higher molecular weight dissolved solids such as residual protein and yeast components (as well as amino acids, simple sugars, organically available nitrogen, for example). The filtered protein and/or yeast components/nutrients can be sent onward in the process to be recovered, such as in the protein meal product and/or the resulting DDG(S), for example, or optionally sent back to fermentation.

With respect to the membrane filtration device 90, a wide variety of filter media as the membrane filtration devices 90 are suitable for use. In one example, any membrane filter with a pore size suitably small enough to exclude the passing of solids such as, for example, protein solids. Such membrane filters can include polymer membranes, sintered metal filters, coated filters, or ceramic filters, and the like. In one example, the pore size required for this type of application falls into the area normally described as microfiltration. In another example, ultrafiltration, which is the next step smaller in pore size, could also be used if the microfiltration method does not desirably reject/separate enough of the protein and/or yeast component solids that needs to be recycled for recovery. In other words, the membrane filtration device 90 can be a microfilter, an ultrafilter, or a nanofilter. In one example, there may be more than one membrane filtration device 90, which may be the same or different type of device and can be arranged in series or parallel. In one embodiment, the membrane filtration device 90 includes a microfiltration device followed by an ultrafiltration device. The membrane filtration device 90 also could be set up for either cross flow or perpendicular flow depending on the nature of the media selected. A cross flow installation typically will have a longer run time between filter changes out due to the scouring nature of flow across the filter surface.

With continuing reference to FIG. 4, the enzyme filtrate, with its active enzyme(s), from the membrane filtration device 90 then can be recycled back into the system, as shown, and reused again such as by being recombined with the solids from the first decanter centrifuge 84 to break down the yeast therein. And the retentate or solids portion, which includes residual protein and yeast components, then can be recombined with the solids stream, which includes protein and yeast components/fragments, from the second decanter centrifuge 88 and the centrate from the first decanter centrifuge 84. The yeast fragments can be later removed from the protein in the combined centrate and solids stream via the subsequent protein separation methods/apparatuses, as described above in FIG. 3, which can provide a more desirable/increased protein purity in any final or resulting protein grain meal product.

Due to the addition of the membrane filtration device 90, the dewatered (and optionally dried) protein product, which excludes much of the yeast, can define a high protein meal that includes, for example, at least 40 wt % protein on a dry basis and which may be sold as pig or chicken feed. In another embodiment, the high protein meal includes at least 50 wt % protein on a dry basis. In another embodiment, the high protein meal includes at least 52 wt % protein on a dry basis. In yet another embodiment, the high protein meal includes at least 55 wt % protein on a dry basis. In still another embodiment, the high protein meal includes about 60, 70, or 80 wt % protein on a dry basis. In another embodiment, the high protein meal can include from about 40 wt % to about 60, 70, or 80 wt % protein. In another embodiment, the high protein meal can include from about 50 wt % to about 60 wt % protein, about 52 wt % to about 60 wt % protein, or about 55 wt % to about 60 wt % protein on a dry basis.

The amount of yeast in the protein of the high protein meal here can vary and, in one example, can similarly include 24% of the total high protein meal. The range of the amount of yeast in the high protein meal generally also similarly can be from about 5% to about 30% depending upon the type of feedstock and other factors, including type of separation machinery, and the like, as would be understood by those skilled in the art. Generally, the purity of the high protein meal can be increased by enzyme treatment of yeast. In one example, the purity of the high protein meal similarly can be increased by 2% relative to a process without the enzyme treatment, for example, such as the process of FIG. 2. In another example, the purity of the high protein meal can be increased by 5%. In yet another example, the purity of the high protein meal can be increased by 10%. Again, while the removal of yeast cells/yeast cell components from the protein stream may reduce the overall yield of the high protein meal, the removal can desirably increase the protein purity of the final product. That is, because the high protein meal now excludes yeast, the yeast is no longer a hindrance to realizing higher protein purity in any final protein meal product.

While the present invention has been illustrated by the description of one or more embodiments thereof, and while the embodiments have been described in considerable detail, they are not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicant's general inventive concept.

What is claimed is:

1. A method for increasing the purity of a protein product obtained from a whole stillage byproduct produced in a biochemical production process comprising:
separating a whole stillage byproduct into a fiber portion and a filtrate, which includes protein particles and yeast cells;
subjecting the filtrate to one or more enzymes to break-up the yeast cells into yeast fragments;
separating the enzymatically treated filtrate, including the protein particles and yeast fragments, via density, into a protein portion, including protein particles, and a water soluble solids portion, including yeast fragments and free oil; and
drying the protein portion to define a high protein grain meal that includes from 40 wt % protein to 80 wt % protein on a dry basis.

2. The method of claim 1 further comprising separating the filtrate, via density, into a solids portion, including the protein particles and yeast cells, and a centrate, and
subjecting the solids portion to the one or more enzymes to break-up the yeast cells into yeast fragments, and separating the enzymatically treated solids portion, including the protein particles and yeast fragments, via density, into the protein portion, including protein particles, and the water soluble solids portion, including yeast fragments and free oil.

3. The method of claim 2 wherein the centrate is combined with the enzymatically treated solids portion prior to separating the enzymatically treated solids portion.

4. The method of claim 1 wherein at least a portion of the one or more enzymes are recovered and recycled from a later step in the method.

5. The method of claim 1 wherein the one or more enzymes are selected from an amylase, alpha-amylase, glucoamylase, fungal, phytase, protease, cellobiose, cellulase, hemicellulase, xylanase, glucanase, beta-glucanase, transglutaminase, zymolyase, or combinations thereof.

6. The method of claim 1 further comprising separating the enzymatically treated filtrate, via density, into a solids portion, including the protein particles and yeast fragments, and a centrate, including the one or more enzymes, which are recycled back into the method and reused as at least a portion of the one or more enzymes; and
separating the solids portion, including the protein particles and yeast fragments, via density, into a protein portion, including protein particles, and a water soluble solids portion, including yeast fragments and free oil.

7. The method of claim 6 further comprising separating the centrate, via filtration, into a retentate, which includes residual protein particles and yeast fragments, and a recycled enzyme filtrate, including the one or more enzymes, which are recycled back into the method and reused as at least a portion of the one or more enzymes.

8. The method of claim 7 wherein the retentate is combined with the solids portion.

9. The method of claim 7 wherein separating the centrate, via filtration, into a retentate and a recycled enzyme filtrate comprises separating the centrate, via membrane filtration.

10. The method of claim 9 wherein the membrane filtration includes micro, ultra, or nanofiltration.

11. The method of claim 1 further comprising, along with subjecting the filtrate to the one or more enzymes, subjecting the filtrate to an acid or base to control pH of the filtrate and/or controlling the temperature of the filtrate.

12. The method of claim 1 further comprising after separating the enzymatically treated filtrate, separately adding a wash water to the separated protein portion followed by dewatering the protein portion to provide a liquid fraction and a protein wet cake fraction, and drying the protein wet cake fraction to define a high protein grain meal that includes from 40 wt % protein to 60 wt % protein on a dry basis.

13. The method of claim 12 further comprising utilizing the liquid fraction in a step earlier in the method.

14. The method of claim 1 further comprising subjecting the water soluble solids portion to evaporation via an evaporator followed by separating free oil from the water soluble solids portion to provide an oil portion.

15. A method for increasing the purity of a protein product obtained from a whole stillage byproduct produced in a biochemical production process comprising:
separating a whole stillage byproduct, via filtration, into a fiber portion and a filtrate, which includes protein particles and yeast cells;
separating the filtrate, via density, into a first solids portion, including the protein particles and yeast cells, and a first centrate;
subjecting the first solids portion to one or more enzymes to break-up the yeast cells into yeast fragments;
separating the enzymatically treated first solids portion, via density, into a second solids portion, including the protein particles and yeast fragments, and a second centrate, including the one or more enzymes, which are recycled back into the method and reused as at least a portion of the one or more enzymes;
separating the second solids portion, including the protein particles and yeast fragments, via density, into a protein portion, including protein particles, and a water soluble solids portion, including yeast fragments and free oil; and
drying the protein portion to define a high protein grain meal that includes from 40 wt % protein to 80 wt % protein on a dry basis.

16. The method of claim 15 wherein the one or more enzymes are selected from an amylase, alpha-amylase, glucoamylase, fungal, phytase, protease, cellobiose, cellulase, hemicellulase, xylanase, glucanase, beta-glucanase, transglutaminase, zymolyase, or combinations thereof.

17. The method of claim 15 further comprising separating the second centrate, via membrane filtration, into a retentate, which includes residual protein particles and yeast fragments, and a recycled enzyme filtrate, including the one or more enzymes, which are recycled back into the method and reused as at least a portion of the one or more enzymes.

18. The method of claim 15 further comprising, along with subjecting the first solids portion to the one or more enzymes, subjecting the first solids portion to an acid or base to control pH of the first solids portion and/or controlling the temperature of the first solids portion.

19. The method of claim 15 further comprising after separating the second solids portion, including the protein particles and yeast fragments, via density, into a protein portion, including protein particles, and a water soluble solids portion, including yeast fragments and free oil, separately adding a wash water to the separated protein portion followed by dewatering the protein portion to provide a liquid fraction and a protein wet cake fraction, and drying the protein wet cake fraction to define a high protein meal that includes from 40 wt % protein to 60 wt % protein on a dry basis.

20. A system for increasing the purity of a protein product obtained from a whole stillage byproduct produced in a biochemical production process comprising:
a first apparatus that is configured to receive a whole stillage byproduct produced in a biochemical production process, wherein the first apparatus separates the whole stillage byproduct into a fiber portion and a filtrate, which includes protein particles and yeast cells;
an enzyme treatment tank that is situated after the first apparatus and that is configured to receive one or more enzymes and the filtrate from the first apparatus, and whereat the yeast cells in the filtrate are broken-up into yeast fragments via the one or more enzymes;
a second apparatus that is situated after the enzyme treatment tank and that is configured to receive the enzymatically treated filtrate from the enzyme treatment tank, wherein the second apparatus separates the enzymatically treated filtrate, including the protein particles and yeast fragments, via density, into a protein portion, including protein particles, and a water soluble solids portion, including yeast fragments and free oil; and
a dryer that is situated after the second apparatus and that is configured to receive and dry the protein portion to define a high protein grain meal that includes from 40 wt % protein to 80 wt % protein on a dry basis.

21. The system of claim 20 wherein the first apparatus is a pressure screen and the second apparatus is a centrifuge.

22. The system of claim 20 further comprising a third apparatus that is situated between the first apparatus and the enzyme treatment tank and that is configured to receive the filtrate from the first apparatus, the third apparatus separates the filtrate, via density, into a solids portion, including the protein particles and yeast cells, and a centrate, and
wherein the enzyme treatment tank is configured to receive the solids portion from the first apparatus, and whereat the solids portion is subjected to one or more enzymes to break-up the yeast cells into yeast fragments, and
wherein the second apparatus is configured to receive the enzymatically treated solids portion from the enzyme treatment tank and separates the enzymatically treated solids portion, including the protein particles and yeast fragments, via density, into a protein portion, including protein particles, and a water soluble solids portion, including yeast fragments and free oil.

23. The system of claim 20 further comprising a third apparatus that is situated between the enzyme treatment tank and the second apparatus and that is configured to receive the enzymatically treated filtrate from the enzyme treatment tank, the third apparatus separates the enzymatically treated filtrate, via density, into a solids portion, including the protein particles and yeast fragments, and a centrate, including the one or more enzymes, wherein the centrate is recycled back into the system and reused as at least a portion of the one or more enzymes at the enzyme treatment tank, and
wherein the second apparatus receives the solids portion from the third apparatus and separates the solids portion, including the protein particles and yeast fragments, via density, into a protein portion, including protein particles, and a water soluble solids portion, including yeast fragments and free oil.

24. The system of claim 23 further comprising a membrane filtration device that is situated after the third apparatus and that is configured to receive the centrate from the third apparatus, the membrane filtration device separates, via filtration, the centrate into a retentate, which includes residual protein particles and yeast fragments, and a recycled enzyme filtrate, including the one or more enzymes, which are recycled back into the system and reused as at least a portion of the one or more enzymes at the enzyme treatment tank.

25. The system of claim 20 further comprising a dewatering device that is situated between the second apparatus and the dryer and that receives the protein portion from the second apparatus, the dewatering device is configured to dewater the protein portion to provide a liquid fraction and a protein wet cake fraction, and
wherein the dryer receives and dries the protein wet cake fraction to define a high protein grain meal that includes from 40 wt % protein to 60 wt % protein on a dry basis.
